(19) United States
(12) Patent Application Publication
(10) Pub. No.: US 2010/0022592 A1
(43) Pub. Date: Jan. 28, 2010

(54) THIAZOLE DERIVATIVES AS MODULATORS OF G PROTEIN-COUPLED RECEPTORS

(75) Inventors: Robert Epple, San Diego, CA (US); Mihai Azimioara, San Diego, CA (US); Christopher Cow, San Diego, CA (US); Ross Russo, Encinitas, CA (US); Victor Nikulin, Carlsbad, CA (US); Gerald Lelais, San Diego, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 12/527,826

(22) PCT Filed: Jan. 7, 2008
(86) PCT No.: PCT/US2008/050434

§ 371 Date: Oct. 14, 2009

Related U.S. Application Data
(60) Provisional application No. 60/891,184, filed on Feb. 22, 2007.

(51) Int. Cl. A61K 31/427; C07D 417/10; C07D 417/14; C12N 5/00; A61K 31/454; A61K 31/4439; A61P 3/10

(52) U.S. Cl. 514/326; 548/202; 546/270.4; 546/209; 435/375; 514/342; 514/365

(57) ABSTRACT

The invention provides compounds of formula (IA) or (IB) and pharmaceutical compositions thereof, which are useful for modulating G protein-coupled receptor 120 (GPR120), and methods for using such compounds to treat, ameliorate or prevent a condition associated with abnormal or deregulated GPR120.

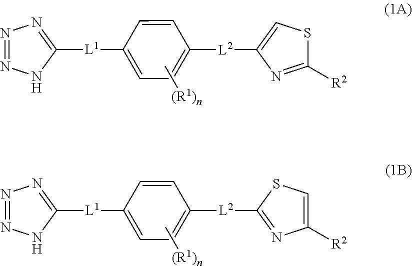

### CROSS-REFERENCE TO RELATED APPLICATIONS

This application claims the benefit of U.S. provisional application Ser. No. 60/891,184, filed Feb. 22, 2007, which is incorporated herein by reference in its entirety.

### TECHNICAL FIELD

The present invention generally relates to G protein-coupled receptors.

### BACKGROUND ART

G-protein coupled receptors (GPCRs) constitute a major class of proteins responsible for transducing a signal within a cell. Upon binding of a ligand to an extracellular portion of a GPCR, a signal is transduced within the cell that results in a change in a biological or physiological property of the cell. GPCRs, along with G-proteins and effectors (intracellular enzymes and channels modulated by G-proteins), are the components of a modular signaling system that connects the state of intracellular second messengers to extracellular inputs.

GPCR genes and gene-products are potential causative agents of disease (Spiegel et al., J. Clin. Invest. 92:1119 1125 (1993)). For example, specific defects in the rhodopsin gene and the V2 vasopressin receptor gene have been shown to cause various forms of retinitis pigmentosum (Nathans et al., Annu. Rev. Genet. 26:403 424 (1992)), and nephrogenic diabetes insipidus (Holtzman et al., Hum. Mol. Genet. 2:1201 1204 (1993)). These receptors are important to both the central nervous system and peripheral physiological processes.

G protein coupled receptor 120 (GPR120) is an orphan G protein-coupled receptor that is abundantly expressed in intestine, and functions as a receptor for unsaturated long-chain free fatty acids (FFAs). (Hirasawa et al., Nature Medicine 11:90-94 (2005)). Stimulation of GPR120 by FFAs has been reported to promote the secretion of glucagon-like peptide-1 (GLP-1) and increase circulating insulin, and to activate the extracellular signal-regulated kinase (ERK) cascade. (Katsuma et al., J. Biol. Chem. 280:19507-19515 (2005)). Peripherally, GLP-1 affects gut motility, and inhibits gastric acid and glucagon secretion. In the central nervous system, GLP-1 induces satiety, leading to reduced weight gain. In the pancreas, GLP-1 induces expansion of insulin-secreting β-cell mass, in addition to the augmentation of glucose-stimulated insulin secretion. (MacDonald et al., Diabetes 51:Supp. 3 S434-S442 (2002)).

Given the significance of GLP-1 as a potent insulinotropic incretin and in appetite and feeding control, GPR120 is a promising target for the treatment of diabetes, obesity and other eating disorders. Because of the importance of GPCRs as targets for drug action and development, there remains a need for the development of agents which modulate GPCR function.

### DISCLOSURE OF THE INVENTION

The invention provides compounds, pharmaceutical compositions and methods of using such compounds for modulating G protein-coupled receptors, more particularly GPR120.

In one aspect, the present invention provides compounds comprising Formula (1):

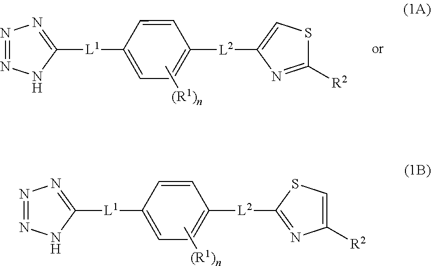

or pharmaceutically acceptable salts, hydrates, solvates and prodrugs thereof, wherein:

L¹ is a bond or —CR³₂—O—;

L² is a bond or NR³;

R¹ is OR, or an optionally substituted C₁₋₆ alkyl, C₂₋₆ alkenyl or C₃₋₆ alkynyl;

R² is NR³—(CR³₂)ₘ—R⁴ or R⁴;

R³ is H or C₁₋₆ alkyl;

R⁴ is C₃₋₇ cycloalkyl, aryl, heteroaryl or heterocyclic ring containing N, O or S, each of which is optionally substituted with halo, hydroxyl, nitro, OR, SR, NR₂, NR³—(CR³₂)ₘ—, or an optionally substituted C₁₋₆ alkyl, C₂₋₆ alkenyl or C₃₋₆ alkynyl; or

R³ and —(CR³₂)ₘ—R⁴ together with N in NR³—(CR³₂)ₘ—R⁴ form an optionally substituted heterocyclic ring;

each R is H, an optionally substituted C₁₋₆ alkyl or —(CR³₂)ₘ—R⁴; and

m and n are independently 0-4.

In one embodiment, the compounds of the invention comprise Formula (2A) or (2B):

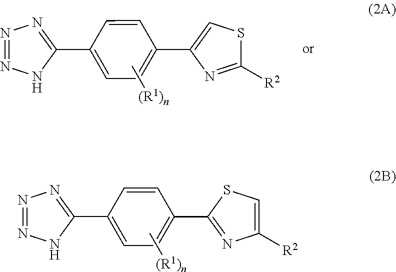

wherein each n is 0; and

R¹ and R² are as defined in Formula (1A) and (1B).

In the above Formula (1A), (1B), (2A) and (2B), R² may be an optionally substituted phenyl, piperidinyl or pyridyl.

In another embodiment, the compounds of the invention comprise Formula (3A) or (3B):

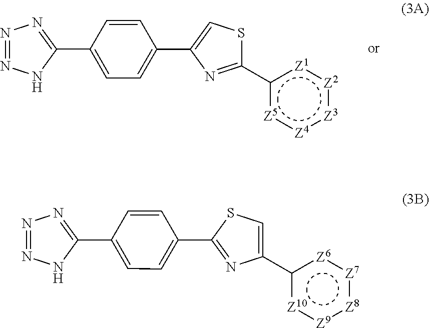

wherein Z¹, Z², Z³, Z⁴, Z⁵, Z⁶, Z⁷, Z⁸, Z⁹ and Z¹⁰ are independently CR⁵ or N;

R⁵ is H halo, hydroxyl, nitro, OR, SR, NR₂, NR³—(CR³₂)ₘ—R⁴, or an optionally substituted C₁₋₆ alkyl, C₂₋₆ alkenyl or C₃₋₆ alkynyl; and

R, R³, R⁴ and m are as defined in Formula (1A) and (1B).

In the above Formula (3A) or (3B), each Z¹, Z², Z³, Z⁴, Z⁵, Z⁶, Z⁷, Z⁸, Z⁹ and Z¹⁰ may be CR⁵. In other examples, one of Z¹, Z², Z³, Z⁴, Z⁵, Z⁶, Z⁷, Z⁸, Z⁹ and Z¹⁰ is N.

In yet another embodiment, the compounds of the invention comprise Formula (4A) or (4B):

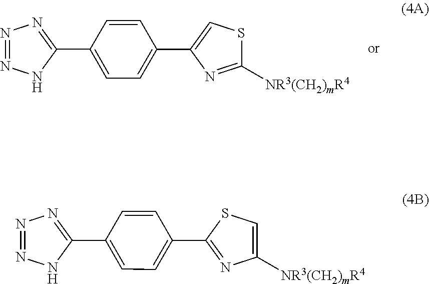

wherein R³ is H or C₁₋₆ alkyl;

R⁴ is a 5-6 membered aryl or heteroaryl; or

R³ and —(CR³₂)ₘ—R⁴ together with N in NR³—(CR³₂)ₘ—R⁴ form an optionally substituted 4-7 membered heterocyclic ring; and

m is 0 or 1.

In the above Formula (4A) and (4B), R⁴ may be phenyl or R³ and —(CR³₂)ₘ—R⁴ together with N in NR³—(CR³₂)ₘ—R⁴ may form an optionally substituted piperidinyl.

In another aspect, the present invention provides pharmaceutical compositions comprising a compound of Formula (1A), (1B), (2A), (2B), (3A), (3B), (4A) or (4B), and a pharmaceutically acceptable excipient.

In yet another aspect, the invention provides methods for modulating G protein-coupled receptor 120 (GPR120), comprising administering to a cell or tissue system or to a mammalian subject, a therapeutically effective amount of a compound comprising Formula (1A), (1B), (2A), (2B), (3A), (3B), (4A) or (4B), or pharmaceutically acceptable salts or pharmaceutical compositions thereof, and optionally in combination with a second therapeutic agent, thereby modulating said GPR120. In particular embodiments, compounds of the invention are GPR120 agonists.

The invention also provides methods for preventing, ameliorating or treating a condition mediated by G protein-coupled receptor 120 (GPR120), comprising administering to a cell or tissue system or to a mammalian subject, an effective amount of a compound comprising Formula (1A), (1B), (2A), (2B), (3A), (3B), (4A) or (4B), or pharmaceutically acceptable salts or pharmaceutical compositions thereof, and optionally with a second therapeutic agent, thereby treating said condition.

Furthermore, the present invention provides the use of a compound of Formula (1A), (1B), (2A), (2B), (3A), (3B), (4A) or (4B), for treating a condition mediated by G protein-coupled receptor 120 (GPR120). The present invention also provides the use of a compound of Formula (1A), (1B), (2A), (2B), (3A), (3B), (4A) or (4B) in the manufacture of a medicament for treating a condition mediated by GPR120.

Examples of conditions which may be ameliorated or treated using the compounds of the invention include but are not limited to diabetes such as diabetes mellitus, dyslipidemia such as hyperlipidemia, obesity or anorexia.

### DEFINITIONS

“Alkyl” refers to a moiety and as a structural element of other groups, for example halo-substituted-alkyl and alkoxy, and may be straight-chained or branched. An optionally substituted alkyl, alkenyl or alkynyl as used herein may be optionally halogenated (e.g., CF₃), or may have one or more carbons that is substituted or replaced with a heteroatom, such as NR, O or S (e.g., —OCH₂CH₂O—, alkylthiols, thioalkoxy, alkylamines, etc).

“Aryl” refers to a monocyclic or fused bicyclic aromatic ring containing carbon atoms. For example, aryl may be phenyl or naphthyl. “Arylene” means a divalent radical derived from an aryl group.

“Heteroaryl” as used herein is as defined for aryl above, where one or more of the ring members is a heteroatom. Examples of heteroaryls include but are not limited to pyridyl, indolyl, indazolyl, quinoxalinyl, quinolinyl, benzofuranyl, benzopyranyl, benzothiopyranyl, benzo[1,3]dioxole, imidazolyl, benzo-imidazolyl, pyrimidinyl, furanyl, oxazolyl, isoxazolyl, triazolyl, tetrazolyl, pyrazolyl, thienyl, etc.

A “carbocyclic ring” as used herein refers to a saturated or partially unsaturated, monocyclic, fused bicyclic or bridged polycyclic ring containing carbon atoms, which may optionally be substituted, for example, with ═O. Examples of carbocyclic rings include but are not limited to cyclopropyl, cyclobutyl, cyclopentyl, cyclohexyl, cyclopropylene, cyclohexanone, etc.

A “heterocyclic ring” as used herein is as defined for a carbocyclic ring above, wherein one or more ring carbons is a heteroatom. For example, a heterocyclic ring may contain N, O, S, —N═, —S—, —S(O), —S(O)₂—, or —NR— wherein R may be hydrogen, C₁₋₄alkyl or a protecting group. Examples of heterocyclic rings include but are not limited to morpholino, pyrrolidinyl, pyrrolidinyl-2-one, piperazinyl, piperidinyl, piperidinylone, 1,4-dioxa-8-aza-spiro[4.5]dec-8-yl, etc.

Unless otherwise indicated, when a substituent is deemed to be “optionally substituted,” it is meant that the substituent is a group that may be substituted with one or more group(s) individually and independently selected from, for example, an optionally halogenated alkyl, alkenyl, alkynyl, alkoxy, alkylamine, alkylthio, alkynyl, amide, amino, including mono- and di-substituted amino groups, aryl, aryloxy, arylthio, carbonyl, carbocyclic, cyano, cycloalkyl, halogen, heteroalkyl, heteroalkenyl, heteroalkynyl, heteroaryl, heterocyclic, hydroxy, isocyanato, isothiocyanato, mercapto, nitro, O-carbamyl, N-carbamyl, O-thiocarbamyl, N-thiocarbamyl, C-amido, N-amido, S-sulfonamido, N-sulfonamido, C-carboxy, O-carboxy, perhaloalkyl, perfluoroalkyl, silyl, sulfonyl, thiocarbonyl, thiocyanato, trihalomethanesulfonyl, and the protected compounds thereof. The protecting groups that may form the protected compounds of the above substituents are known to those of skill in the art and may be found in references such as Greene and Wuts, Protective Groups in Organic Synthesis, 3^{rd} Ed., John Wiley & Sons, New York, N.Y., 1999, and Kocienski, Protective Groups, Thieme Verlag, New York, N.Y., 1994, which are incorporated herein by reference in their entirety.

The terms “co-administration” or “combined administration” or the like as used herein are meant to encompass administration of the selected therapeutic agents to a single patient, and are intended to include treatment regimens in which the agents are not necessarily administered by the same route of administration or at the same time.

The term “pharmaceutical combination” as used herein refers to a product obtained from mixing or combining active ingredients, and includes both fixed and non-fixed combinations of the active ingredients. The term “fixed combination” means that the active ingredients, e.g. a compound of Formula (1) and a co-agent, are both administered to a patient simultaneously in the form of a single entity or dosage. The term “non-fixed combination” means that the active ingredients, e.g. a compound of Formula (1) and a co-agent, are both administered to a patient as separate entities either simultaneously, concurrently or sequentially with no specific time limits, wherein such administration provides therapeutically effective levels of the active ingredients in the body of the patient. The latter also applies to cocktail therapy, e.g. the administration of three or more active ingredients.

The term “therapeutically effective amount” means the amount of the subject compound that will elicit a biological or medical response in a cell, tissue, organ, system, animal or human that is being sought by the researcher, veterinarian, medical doctor or other clinician.

The term “administration” and or “administering” of the subject compound should be understood to mean as providing a compound of the invention including a pro-drug of a compound of the invention to the individual in need of treatment.

As used herein, the terms “treat”, “treating” and “treatment” refer to a method of alleviating or abating a disease and/or its attendant symptoms.

### MODES OF CARRYING OUT THE INVENTION

The invention provides compounds, pharmaceutical compositions and methods of using such compounds for modulating G protein-coupled receptors, more particularly GPR120.

In one aspect, the present invention provides compounds of Formula (1):

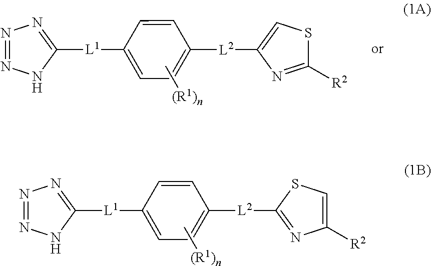

or pharmaceutically acceptable salts, hydrates, solvates and prodrugs thereof, wherein:

L¹ is a bond or —CR³₂—O—;

L² is a bond or NR³;

R¹ is OR, or an optionally substituted C₁₋₆ alkyl, C₂₋₆ alkenyl or C₃₋₆ alkynyl;

R² is NR³—(CR³₂)ₘR⁴ or R⁴;

R³ is H or C₁₋₆ alkyl;

R⁴ is C₃₋₇ cycloalkyl, aryl, heteroaryl or heterocyclic ring containing N, O or S, each of which is optionally substituted with halo, hydroxyl, nitro, OR, SR, NR₂, NR³—(CR³₂)ₘ—, or an optionally substituted C₁₋₆ alkyl, C₂₋₆ alkenyl or C₃₋₆ alkynyl; or

R³ and —(CR³₂)ₘ—R⁴ together with N in NR³—(CR³₂)ₘ—R⁴ form an optionally substituted heterocyclic ring;

each R is H, an optionally substituted C₁₋₆ alkyl or —(CR³₂)ₘ—R⁴; and

m and n are independently 0-4.

In another embodiment, the compounds of the invention comprise Formula (2A) or (2B):

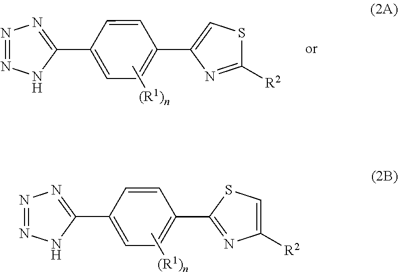

wherein each n is 0; and

R¹ and R² are as defined in Formula (1A) and (1B).

In yet another embodiment, the compounds of the invention comprise Formula (3A) or (3B):

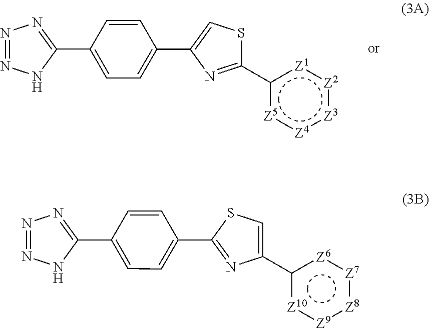

wherein Z¹, Z², Z³, Z⁴, Z⁵, Z⁶, Z⁷, Z⁸, Z⁹ and Z¹⁰ are independently CR⁵ or N; and

R⁵ is H halo, hydroxyl, nitro, OR, SR, NR₂, NR³—(CR³₂)ₘ—R⁴, or an optionally substituted C₁₋₆ alkyl, C₂₋₆ alkenyl or C₃₋₆ alkynyl; and

R, R³, R⁴ and m are as defined in Formula (1A) and (1B).

In yet another embodiment, the compounds of the invention comprise Formula (4A) or (4B):

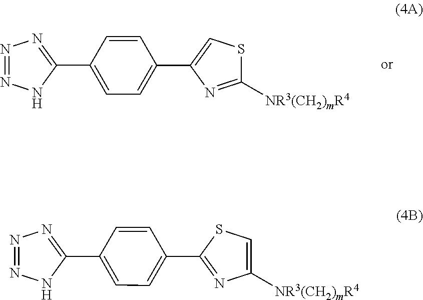

wherein R³ is H or C₁₋₆ alkyl;

R⁴ is a 5-6 membered aryl or heteroaryl; or

R³ and —(CR³₂)ₘ—R⁴ together with N in NR³—(CR³₂)ₘ—R⁴ form an optionally substituted 4-7 membered heterocyclic ring; and

m is 0 or 1.

The present invention also includes all suitable isotopic variations of the compounds of the invention, or pharmaceutically acceptable salts thereof. An isotopic variation of a compound of the invention or a pharmaceutically acceptable salt thereof is defined as one in which at least one atom is replaced by an atom having the same atomic number but an atomic mass different from the atomic mass usually found in nature. Examples of isotopes that may be incorporated into the compounds of the invention and pharmaceutically acceptable salts thereof include but are not limited to isotopes of hydrogen, carbon, nitrogen and oxygen such as ²H, ³H, ¹¹C, ¹³C, ¹⁴C, ¹⁵N, ¹⁷O, ¹⁸O, ³⁵S, ¹⁸F, ³⁶Cl and ¹²³I. Certain isotopic variations of the compounds of the invention and pharmaceutically acceptable salts thereof, for example, those in which a radioactive isotope such as ³H or ¹⁴C is incorporated, are useful in drug and/or substrate tissue distribution studies. In particular examples, ³H and ¹⁴C isotopes may be used for their ease of preparation and detectability. In other examples, substitution with isotopes such as ²H may afford certain therapeutic advantages resulting from greater metabolic stability, such as increased in vivo half-life or reduced dosage requirements. Isotopic variations of the compounds of the invention or pharmaceutically acceptable salts thereof can generally be prepared by conventional procedures using appropriate isotopic variations of suitable reagents.

The compounds of the invention may be useful for modulating G protein-coupled receptors (GPCRs), particularly GPR120. For example, compounds of the invention may be useful as GPR120 agonists. Compounds of the invention may also be useful for treating conditions mediated by GPR120, including but are not limited to diabetes such as diabetes mellitus, dyslipidemia such as hyperlipidemia, obesity or anorexia.

Pharmacology and Utility

Compounds of the invention may modulate G protein-coupled receptors, and as such, are useful for treating diseases or disorders in which GPCR contribute to the pathology and/or symptomology of the disease. More particularly, the compounds of the invention may be used to prevent, ameliorate or treat a condition mediated by G protein-coupled receptor 120 (GPR120).

Examples of conditions mediated by GPR120 include but are not limited to obesity, diabetes, hyperphagia, endocrine abnormalities, triglyceride storage disease, Bardet-Biedl syndrome, Lawrence-Moon syndrome, Prader-Labhart-Willi syndrome, anorexia, and cachexia. In particular examples, obesity is defined as a body mass index (BMI) of 30 kg/m or more (National Institute of Health, Clinical Guidelines on the Identification, Evaluation, and Treatment of Overweight and Obesity in Adults (1998)). However, the compounds of the invention may be used to prevent, ameliorate or treat a condition characterized by a body mass index (BMI) of 25 kg/m or more, 26 kg/m or more, 27 kg/m or more, 28 kg/m or more, 29 kg/m or more, 29.5 kg/m or more, or 29.9 kg/m or more, all of which are typically referred to as overweight (National Institute of Health, Clinical Guidelines on the Identification, Evaluation, and Treatment of Overweight and Obesity in Adults (1998)).

In some embodiments, the compounds of the invention may be useful as an agent for regulating glycerol production from adipocytes, an agent for regulating blood glycerol, an agent for regulating lipolysis, an insulin resistance regulating agent, a stress regulating agent, an agent for regulating adrenocorticotropic hormone (ACTH) secretion, an agent for regulating growth hormone secretion, and an agent for regulating glucagon-like peptide-1 (GLP-1) secretion.

Compounds of the invention that are GPR120 agonists, or that potentiate the binding affinity of free fatty acids to GPR120, may be useful as an agent for suppressing glycerol production from adipocytes, an agent for lowering blood glycerol, an agent for suppressing lipolysis, an agent for suppressing insulin resistance, a stress regulating agent, an adrenocorticotropic hormone (ACTH) secretion suppressing agent, a growth hormone secretion suppressing agent and a glucagon-like peptide-1 (GLP-1) secretion promoting agent. In particular examples, the GPR agonists useful as an adrenocorticotropic hormone (ACTH) secretion suppressing agent may be useful for preventing/treating related diseases, such as ACTH-producing tumor, Cushing's disease, infectious disease, secondary adrenocortical insufficiency, peptic ulcer, diabetes mellitus, mental disorder, cataract, glaucoma, tuberculous disease, hypertension, Cushing's syndrome (e.g., central obesity, edema, hypertension, menstrual disorder, extensive stretch mark, hirsutism, diabetes mellitus, full moon face, osteoporosis, hemorrhagic diathesis, mental disorder (e.g., depression, anxiety), muscular atrophy, loss of muscle strength, hypokalemia, hypercholesterolemia, impaired glucose resistance, leukocytosis), adrenocortical atrophy, etc.

Compounds of the invention that are GPR120 antagonists, or that reduce the binding affinity of free fatty acids to GPR120, may be useful as an agent for promoting glycerol production from adipocytes, an agent for increasing blood glycerol, an agent for promoting lipolysis, an agent for promoting insulin resistance, a stress regulating agent, an agent for promoting adrenocorticotropic hormone (ACTH) secretion, an agent for promoting growth hormone secretion and an agent for suppressing glucagon-like peptide-1 (GLP-1) secretion). In particular examples, the GPR120 antagonists useful as an agent for promoting adrenocorticotropic hormone (ACTH) secretion may be useful for preventing/treating connective tissue diseases (e.g., chronic articular rheumatism, systemic lupus erythematosus, polymyositis, rheumatic fever, scleroderma), kidney diseases (e.g., nephrosis), respiratory diseases (e.g., bronchial asthma, pulmonary tuberculous pleuritis, sarcoidosis, diffuse interstitial pneumonia), alimentary diseases (e.g., ulcerative colitis, cholestatic acute hepatitis, fulminant hepatitis, chronic hepatitis, cirrhosis), neuromuscular diseases (e.g., encephalomyelitis, peripheral neuritis, multiple sclerosis, myasthenia gravis, facial paralysis), blood diseases (e.g., hemolytic anemia, agranulocytosis, purpura, aplastic anemia, leukemia, malignant lymphoma), endocrine-metabolic diseases (e.g., acute or chronic adrenocortical insufficiency, adrenogenital syndrome, malignant exopthalmos due to thyroid gland disease, ACTH isolated deficiency), skin diseases (e.g., urticaria, eczema, dermatitis, herpes zoster, psoriasis, drug allergy) or anaphylactic shock, etc.

Compounds of the invention may also be useful as an agent for preventing/treating, for example, diabetes mellitus, impaired glucose tolerance, ketosis, acidosis, diabetic neuropathy, diabetic nephropathy, diabetic retinopathy, hyperlipemia, arteriosclerosis, angina pectoris, myocardial infarction, sexual dysfunction, obesity, pituitary dysfunctions (e.g., hypopituitarism, pituitary dwarfism, diabetes insipidus, acromegaly, Cushing's disease, hyperprolactinemia, syndrome of inappropriate secretion of anti-diuretic hormone), cancer (e.g., colorectal cancer), deficits in memory and learning, pancreatic exhaustion, hypoglycemia, insulin allergy, lipotoxicity, fatty atrophy, cancerous cachexia, hyperinsulinemia, hyperglycemia, disorder caused by high FFA flux, hypertriglyceridemia, fatty liver, dysfunction of heat production, cholelithiasis, eating disorder, anorexia, secretion disorders of intestinal hormones (e.g., cholecystokinin (CCK), gastric inhibitory peptide (GIP), gastrin, glucagon-like peptide-1 (GLP-1), somatostatin, gastrin-releasing peptide, secretin, vasoactive intestinal peptide, motilin, substance P, neurotensin, galanin, neuropeptide Y, enkephalins, peptide YY, etc.) or circulatory diseases. GPR120 agonists may be particularly useful for preventing/treating diabetes mellitus, hyperlipemia, arteriosclerosis, angina pectoris or myocardial infarction, while GPR120 antagonists may be useful for preventing/treating anorexia and obesity, such as obesity with visceral fat accumulation).

Furthermore, compounds of the invention may be useful as an agent for preventing/treating diseases, for example, arteriosclerosis, arteriosclerotic diseases and their secondary diseases [e.g., acute coronary syndrome such as atherosclerosis, peripheral arterial disease, acute myocardial infarction, unstable angina, etc., ischemic heart diseases such as restenosis after percutaneous transluminal coronary angioplasty (PTCA), myocardial infarction, angina pectoris, etc., arteriosclerosis including angiocalcinosis, etc., intermittent claudication, apoplexy (cerebral infarction, cerebral embolism, brain hemorrhage, etc.), lacunar infarction, cerebrovascular dementia, gangrene, glomerulosclerosis, nephropathy, Tangier disease, etc.], vascular lesions in atherosclerosis and their secondary diseases (e.g., coronary heart disease (CHD), cerebral ischemia, etc.), lipid dysbolism and its secondary diseases, etc.

Administration and Pharmaceutical Compositions

In general, compounds of the invention will be administered in therapeutically effective amounts via any of the usual and acceptable modes known in the art, either singly or in combination with one or more therapeutic agents. The compounds of the invention may be mixed with the other therapeutic agent in a fixed pharmaceutical composition, or may be administered separately, before, simultaneously with or after the other therapeutic agent.

Accordingly, the invention encompasses compounds of the invention, which may be used in combination with other therapeutic substances such as therapeutic agents for treating diabetes, diabetic complications, dyslipidemia and more particularly hyperlipidemia; antihypertensive agents, antiobesity agents, diuretics, chemotreating agents, immunotreating agents, immunomodulators, anti-inflammatory agents, antithrombotic agents, therapeutic agents for osteoporosis, antibacterial agents, antifungal agents, antiprotozoal agents, antibiotics, antitussives and expectorant drugs, sedatives, anesthetics, antiulcer agents, tranquilizers, antipsychotic agents, antitumor agents, muscle relaxants, antiepileptics, antidepressants, antiallergic agents, cardiac stimulants, antiarrhythmic agents, vasodilators, vasoconstrictors, narcotic antagonists, vitamins, vitamin derivatives, antiasthmatic agents, antidementia agents, treating agents for pollakiuria or urinary incontinence, therapeutic agents for dysuria, treating agents for atopic dermatitis, therapeutic agents for allergic rhinitis, vasopressors, endotoxin antagonists or antibodies, signal transduction inhibitors, inflammatory mediator effect suppressants, inflammatory mediator effect suppressing antibodies, anti-inflammatory mediator effect suppressants, anti-inflammatory mediator effect suppressing antibodies and the like.

Therapeutic agents for diabetes include but are not limited to insulin preparations (e.g., animal insulin preparations extracted from pancreas of bovine or pig; human insulin preparations genetically synthesized using Escherichia coli or yeast; zinc insulin; protamine zinc insulin; fragment or derivative of insulin (e.g., INS-1, etc.), oral insulin preparation and the like), insulin sensitizers (e.g., pioglitazone or a salt thereof (preferably hydrochloride), troglitazone, rosiglitazone or a salt thereof (preferably maleate), Reglixane (JTT-501), Netoglitazone (MCC-555), YM-440, GI-262570, KRP-297, FK-614, CS-011, (E)-[[[4-[(5-methyl-2-phenyl-4-oxazolyl)methoxy]phenyl]methoxy]imino]benzenebutanoic acid and the like, compounds described in WO 99/58510 (e.g., (E)-4-[4-(5-methyl-2-phenyl-4-oxazolylmethoxy)-benzyloxyimino]-4-phenylbutyric acid), compounds described in WO 01/38325, Tesaglitazar (AZ-242), Ragaglitazar (N,N-622), BMS-298585, ONO-5816, BM-13-1258, LM-4156, MBX-102, LY-519818, MX-6054, LY-510929, Balaglitazone (N,N-2344), T-131 or a salt thereof, THR-0921), -glucosidase inhibitors (e.g., voglibose, acarbose, miglitol, emiglitate, etc.), biguanides (e.g., phenformin, metformin, buformin, etc.), insulin secretagogues [sulfonylurea (e.g., tolbutamide, glibenclamide, gliclazide, chlorpropamide, tolazamide, acetohexamide, glyclopyramide, glimepiride, etc.), repaglinide, senaglinide, mitiglinide or calcium salt hydrate thereof, nateglinide, etc.], GLP-1 receptor agonists [e.g., GLP-1, GLP-IMR agent, N,N-2211, AC-2993 (exendin-4), BIM-51077, Aib(8,35)hGLP-1(7,37)NH2, CJC-1131, etc.], dipeptidyl peptidase IV inhibitors (e.g., NVP-DPP-278, PT-100, P32/98, P93/01, NVP-DPP-728, LAF237, TS-021, etc.), 3 agonists (e.g., CL-316243, SR-58611-A, UL-TG-307, AJ-9677, AZ40140, etc.), amylin agonists (e.g., pramlintide, etc.), phosphotyrosine phosphatase inhibitors (e.g., vanadic acid, etc.), gluconeogenesis inhibitors (e.g., glycogen phosphorylase inhibitors, glucose-6-phosphatase inhibitors, glucagon antagonists, etc.), SGLT (sodium-glucose cotransporter) inhibitors (e.g., T-1095, etc.), 11-hydroxysteroid dehydrogenase inhibitors (e.g., BVT-3498, etc.), adiponectin or agonists thereof, IKK inhibitors (e.g., AS-2868, etc.), leptin resistance improving drugs, somatostatin receptor agonists (compounds described in WO 01/25228, WO 03/42204, compounds described in WO 98/44921, WO 98/45285, WO 99/22735, etc.), glucokinase activators (e.g., Ro-28-1675) and the like.

Therapeutic agents for treating diabetic complications include but are not limited to aldose reductase inhibitors (e.g., Tolrestat, Epalrestat, Zenarestat, Zopolrestat, Fidarestat (SNK-860), Minalrestat (ARI-509), CT-112, etc.), neurotrophic factors and increasing drugs thereof (e.g., NGF, NT-3, BDNF, neurotrophin production-secretion promoters described in WO 01/14372 (e.g., 4-(4-chlorophenyl)-2-(2-methyl-1-imidazolyl)-5-[3-(2-methylphenoxy)propyl]oxazole, etc.) and the like), protein kinase C (PKC) inhibitors (e.g., LY-333531, etc.), AGE inhibitors (e.g., ALT-945, pimagedine, pyratoxanthine, N-phenacylthiazolium bromide (ALT-766), EXO-226, ALT-711, Pyridorin, Pyridoxamine, etc.), active oxygen scavengers (e.g., thioctic acid, etc.), cerebral vasodilators (e.g., tiapuride, etc.), somatostatin receptor agonist (BIM23190), apoptosis signal regulating kinase-1 (ASK-1) inhibitors and the like.

Therapeutic agents for treating hyperlipidemia include but are not limited to statin compounds which are cholesterol synthesis inhibitors (e.g., pravastatin, simvastatin, lovastatin, atorvastatin, fluvastatin, cerivastatin or a salt thereof (e.g., sodium salt, etc.), etc.), squalene synthase inhibitors (e.g., compounds described in WO 97/10224, such as N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid and the like), fibrate compounds (e.g., bezafibrate, clofibrate, simfibrate, clinofibrate, etc.), antioxidants (e.g., lipoic acid, probucol) and the like.

Examples of the antihypertensive agents include but are not limited to angiotensin converting enzyme inhibitors (e.g., captopril, enalapril, delapril, etc.), angiotensin II antagonists (e.g., losartan, candesartan, cilexetil, eprosartan, valsartan, telmisartan, irbesartan, tasosartan, 1-[[2′-(2,5-dihydro-5-oxo-4H-1,2,4-oxadiazol-3-yl)biphenyl-4-yl]methyl]-2-ethoxy-1H-benzimidazole-7-carboxylic acid, etc.), calcium antagonists (e.g., manidipine, nifedipine, amlodipine, efonidipine, nicardipine, etc.), clonidine and the like.

Examples of the antiobesity agents include but are not limited to antiobesity agents acting on the central nervous system (e.g., dexfenfluramine, fenfluramine, phentermine, sibutramine, amfepramone, dexamphetamine, mazindol, phenylpropanolamine, clobenzorex; MCH receptor antagonists (e.g., SB-568849; SNAP-7941; compounds encompassed in WO 01/82925 and WO 01/87834, etc.); neuropeptide Y antagonists (e.g., CP-422935, etc.); cannabinoid receptor antagonists (e.g., SR-141716, SR-147778, etc.); ghrelin antagonists; 11-hydroxysteroid dehydrogenase inhibitors (e.g., BVT-3498, etc.) and the like), pancreatic lipase inhibitors (e.g., orlistat, ATL-962, etc.), 3 agonists (e.g., CL-316243, SR-58611-A, UL-TG-307, AJ-9677, AZ40140, etc.), peptide anorexiants (e.g., leptin, CNTF (Ciliary Neurotropic Factor), etc.), cholecystokinin agonists (e.g., lintitript, FPL-15849, etc.), feeding deterrent (e.g., P-57, etc.) and the like.

Examples of the diuretics include but are not limited to xanthine derivatives (e.g., theobromine sodium salicylate, theobromine calcium salicylate, etc.), thiazide preparations (e.g., ethiazide, cyclopenthiazide, trichloromethiazide, hydrochlorothiazide, hydroflumethiazide, benzyl hydrochlorothiazide, penflutizide, polythiazide, methyclothiazide, etc.), antialdosterone preparations (e.g., spironolactone, triamterene, etc.), carbonate dehydratase inhibitors (e.g., acetazolamide and the like), chlorobenzenesulfonamide preparations (e.g., chlorthalidone, mefruside, indapamide, etc.), azosemide, isosorbide, ethacrynic acid, piretanide, bumetanide, furosemide and the like.

Examples of the chemotreating agents include but are not limited to alkylating agents (e.g., cyclophosphamide, ifosfamide, etc.), metabolic antagonists (e.g., methotrexate, 5-fluorouracil, etc.), antitumor antibiotics (e.g., mitomycin, adriamycin, etc.), plant-derived antitumor agent (e.g., vincristine, vindesine, Taxol, etc.), cisplatin, carboplatin, etoposide and the like.

Examples of the immunotreating agents include but are not limited to microorganism or bacterial components (e.g., muramyl dipeptide derivative, Picibanil, etc.), polysaccharides having immunity potentiating activity (e.g., lentinan, schizophyllan, krestin, etc.), cytokines obtained by genetic engineering techniques (e.g., interferon, interleukin (IL), etc.), colony stimulating factors (e.g., granulocyte colony stimulating factor, erythropoietin, etc.) and the like, with preference given to interleukins such as IL-1, IL-2, IL-12 and the like.

Examples of the anti-inflammatory agents include but are not limited to non-steroidal anti-inflammatory agents such as aspirin, acetaminophen, indomethacin and the like.

Examples of the antithrombotic agents include but are not limited to heparin (e.g., heparin sodium, heparin calcium, dalteparin sodium, etc.), warfarin (e.g., warfarin potassium, etc.), antithrombin drugs (e.g., aragatroban, etc.), thrombolytic agents (e.g., urokinase, tisokinase, alteplase, nateplase, monteplase, pamiteplase, etc.), platelet aggregation suppressors (e.g., ticlopidine hydrochloride, cilostazol, ethyl icosapentate, beraprost sodium, sarpogrelate hydrochloride, etc.) and the like.

Therapeutic agents for treating osteoporosis include but are not limited to alfacalcidol, calcitriol, elcatonin, calcitonin salmon, estriol, ipriflavone, pamidronate disodium, alendronate sodium hydrate, incadronate disodium and the like.

Examples of the vitamins which may be used in combination with the compounds of the invention include but are not limited to vitamin B1, vitamin B12 and the like, and derivatives thereof.

Examples of the antidementia agents include but are not limited to tacrine, donepezil, rivastigmine, galantamine and the like.

Therapeutic agents for pollakiuria or urinary incontinence include but are not limited to flavoxate hydrochloride, oxybutynin hydrochloride, propiverine hydrochloride and the like.

Therapeutic agents for dysuria include but are not limited to acetylcholine esterase inhibitors (e.g., distigmine) and the like.

Furthermore, therapeutic agents having a cachexia-improving effect in animal models and clinical situations, may be used in combination with the compound of the invention. Examples of such therapeutic agents include but are not limited to cyclooxygenase inhibitors (e.g., indomethacin, etc.) [Cancer Research, Vol. 49, pp. 5935-5939, 1989], progesterone derivatives (e.g., megestrol acetate) [Journal of Clinical Oncology, Vol. 12, pp. 213-225, 1994], glucosteroids (e.g., dexamethasone, etc.), metoclopramide agents, tetrahydrocannabinol agents (publications are all as mentioned above), fat metabolism improving agents (e.g., eicosapentanoic acid, etc.) [British Journal of Cancer, Vol. 68, pp. 314-318, 1993], growth hormones, IGF-1, or antibodies to a cachexia-inducing factor such as TNF-, LIF, IL-6, oncostatin M and the like.

Furthermore, glycosylation inhibitors (e.g., ALT-711, etc.), nerve regeneration promoting drugs (e.g., Y-128, VX853, prosaptide, etc.), antidepressants (e.g., desipramine, amitriptyline, imipramine, etc.), antiepileptics (e.g., lamotrigine, Trileptal, Keppra, Zonegran, Pregabalin, Harkoseride, carbamazepine), antiarrhythmic agents (e.g., mexiletine), acetylcholine receptor ligands (e.g., ABT-594), endothelin receptor antagonists (e.g., ABT-627), monoamine uptake inhibitors (e.g., tramadol), narcotic analgesics (e.g., morphine), GABA receptor agonists (e.g., gabapentin, gabapentin MR preparations), 2 receptor agonists (e.g., clonidine), local analgesics (e.g., capsaicin), antianxiety drugs (e.g., benzodiazepines), phosphodiesterase inhibitors (e.g., sildenafil), dopamine receptor agonists (e.g., apomorphine) and the like may also be used in combination with the compounds of the invention.

A therapeutically effective amount may vary widely depending on the severity of the disease, the age and relative health of the subject, the potency of the compound used and other factors. In general, satisfactory results are indicated to be obtained systemically at daily dosages of from about 0.01 to 2.5 mg/kg per body weight. An indicated daily dosage in the larger mammal, e.g. humans, is in the range from about 0.01 mg/kg per body weight to about 100 mg, conveniently administered, e.g. in divided doses up to four times a day or in retard form. Suitable unit dosage forms for oral administration comprise from ca. 0.01 to 50 mg/kg per body weight. For example, a compound of the invention (as an active ingredient) may be orally administered to a patient with hyperlipidemia in about 0.01 to about 30 mg/kg of body weight per day; in some examples, from about 0.1 to about 20 mg/kg of body weight per day; and in other examples, from about 1 to about 20 mg/kg of body weight per day, which may be given at once or in several portions a day.

Compounds of the invention may be administered as pharmaceutical compositions by any conventional route known in the art, such as those described in EP 1688138, incorporated by reference herein in its entirety. For example, compounds of the invention may be administered enterally, e.g., orally, e.g., in the form of tablets or capsules, or parenterally, e.g., in the form of injectable solutions or suspensions, topically, e.g., in the form of lotions, gels, ointments or creams, or in a nasal or suppository form.

Pharmaceutical compositions comprising a compound of the present invention in free form or in a pharmaceutically acceptable salt form in association with at least one pharmaceutically acceptable carrier or diluent may be manufactured in a conventional manner by mixing, granulating or coating methods. For example, oral compositions may be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets, together with c) binders, e.g., magnesium aluminum silicate, starch paste, gelatin, tragacanth, methylcellulose, sodium carboxymethylcellulose and or polyvinylpyrrolidone; and if desired, d) disintegrants, e.g., starches, agar, alginic acid or its sodium salt, or effervescent mixtures; and/or e) absorbents, colorants, flavors and sweeteners. Injectable compositions may be aqueous isotonic solutions or suspensions, and suppositories may be prepared from fatty emulsions or suspensions.

The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier may include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, may be aqueous solutions, ointments, creams or gels well-known in the art. Such may contain solubilizers, stabilizers, tonicity enhancing agents, buffers and preservatives.

The invention also provides kits of a pharmaceutical combinations, comprising a) a first agent comprising a compound of Formula (1A), (1B), (2A), (2B), (3A), (3B), (4A) or (4B), in free form or in pharmaceutically acceptable salt form, and b) at least one co-therapeutic agent. The kit may further comprise instructions for its administration.

Processes for Making Compounds of the Invention

The compounds of the invention may be prepared, following procedures exemplified in the Examples.

Compounds of the invention may also be prepared as a pharmaceutically acceptable acid addition salt by reacting the free base form of the compound with a pharmaceutically acceptable inorganic or organic acid. Alternatively, a pharmaceutically acceptable base addition salt of a compound of the invention may be prepared by reacting the free acid form of the compound with a pharmaceutically acceptable inorganic or organic base. Alternatively, salt forms of the compounds of the invention may be prepared using salts of the starting materials or intermediates.

The free acid or free base forms of the compounds of the invention may be prepared from the corresponding base addition salt or acid addition salt from, respectively. For example a compound of the invention in an acid addition salt form may be converted to the corresponding free base by treating with a suitable base (e.g., ammonium hydroxide solution, sodium hydroxide, and the like). A compound of the invention in a base addition salt form may be converted to the corresponding free acid by treating with a suitable acid (e.g., hydrochloric acid, etc.).

Compounds of the invention in unoxidized form may be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Protected derivatives of the compounds of the invention may be made by means known to those of ordinary skill in the art. A detailed description of techniques applicable to the creation of protecting groups and their removal may be found in T. W. Greene, “Protecting Groups in Organic Chemistry”, 3^{rd} edition, John Wiley and Sons, Inc., 1999.

Compounds of the present invention may be conveniently prepared, or formed during the process of the invention, as solvates (e.g., hydrates). Hydrates of compounds of the present invention may be conveniently prepared by recrystallization from an aqueous/organic solvent mixture, using organic solvents such as dioxin, tetrahydrofuran or methanol.

The invention also provides prodrug derivatives of the compounds of the invention. Conversion of prodrug derivatives to the compounds of the invention may occur under physiological conditions as described in “Pharmaceutical Development”, vol. 7 (Molecular Design), pp. 163-198 (1990), or with a reaction by an enzyme, a gastric acid, etc. in the living body (e.g., conversion by enzymatic oxidation, reduction, hydrolysis, etc.).

Prodrug derivatives of the compounds of the invention may be prepared by methods known to those of ordinary skill in the art (e.g., Saulnier et al., (1994), Bioorganic and Medicinal Chemistry Letters, Vol. 4, p. 1985). For example, appropriate prodrugs may be prepared by reacting a non-derivatized compound of the invention with a suitable carbamylating agent (e.g., 1,1-acyloxyalkylcarbanochloridate, para-nitrophenyl carbonate, or the like). Other examples include compounds of the invention wherein an amino group is substituted with acyl, alkyl, phosphoric acid, etc. (e.g., substitution of an amino group with eicosanyl, alanyl, pentylaminocarbonyl, (5-methyl-2-oxo-1,3-dioxolen-4-yl)methoxycarbonyl, tetrahydrofuranyl, pyrrolidylmethyl, pivaloyloxymethyl, tert-butyl, etc,); a compound wherein a hydroxy group is substituted with acyl, alkyl, phosphoric acid, boric acid, etc. (e.g., substitution of a hydroxyl group with acetyl, palmitoyl, propanoyl, pivaloyl, succinyl, fumaryl, alanyl, dimethylaminomethylcarbonyl, etc.); a compound wherein a carboxyl group is substituted with ester, amide, etc. (e.g., modification of a carboxyl group with ethyl ester, phenyl ester, carboxymethyl ester, dimethylaminomethyl ester, pivaloyloxymethyl ester, ethoxycarbonyloxyethyl ester, phthalidyl ester, (5-methyl-2-oxo-1,3-dioxolen-4-yl)methyl ester, cyclohexyloxycarbonylethyl ester, methyl amide, etc.); etc. In particular examples, the prodrug derivatives are prepared by esterification of a carboxyl group with a C₁₋₆ alkyl group such as methyl, ethyl, tert-butyl and the like.

Compounds of the invention may be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers may be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and may be readily separated by taking advantage of these dissimilarities. The diastereomers may be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture may be found in Jean Jacques, Andre Collet, Samuel H. Wilen, “Enantiomers, Racemates and Resolutions”, John Wiley And Sons, Inc., 1981.

The compounds of the invention may be prepared as exemplified in the Examples, and may optionally involve:

(a) converting a compound of the invention into a pharmaceutically acceptable salt;

(b) converting a salt form of a compound of the invention to a non-salt form;

(c) converting an unoxidized form of a compound of the invention into a pharmaceutically acceptable N-oxide;

(d) converting an N-oxide form of a compound of the invention to its unoxidized form;

(e) resolving an individual isomer of a compound of the invention from a mixture of isomers;

(f) converting a non-derivatized compound of the invention into a pharmaceutically acceptable prodrug derivative; and

(g) converting a prodrug derivative of a compound of the invention to its non-derivatized form.

Insofar as the production of the starting materials is not particularly described, the compounds are known or may be prepared analogously to methods known in the art or as disclosed in the Examples hereinafter. One of skill in the art will appreciate that the synthetic methodologies described herein are only representative of methods for preparation of the compounds of the present invention, and that other well known methods may similarly be used. The present invention is further exemplified, but not limited, by the following Examples that illustrate the preparation of the compounds of the invention.

### Example 1

### 5-(4-(2-(2-Chlorophenyl)thiazol-4-yl)phenyl)-1H-tetrazole

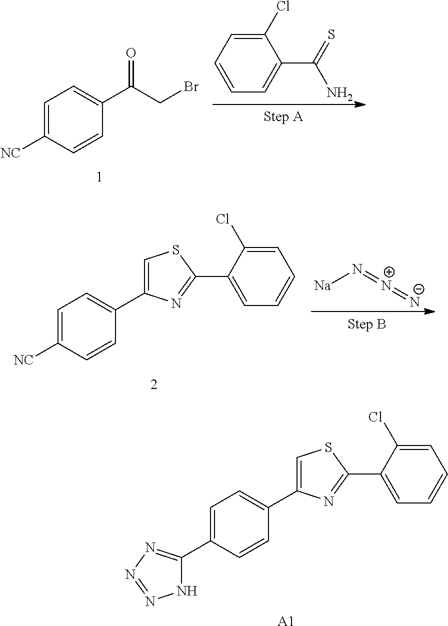

Step A: 4-(2-Bromoacetyl)benzonitrile 1 (0.34 g, 1.5 mmol) and 2-chlorobenzothioamide (0.26 g, 1.5 mmol) are dissolved in EtOH (3 mL) and heated to 150° C. for 3 min using a microwave oven. Dilution with water, filtration, washing with water and drying afforded 4-(2-(2-chlorophenyl)thiazol-4-yl)benzonitrile 2 as a white solid. ¹H-NMR (400 MHz, CDCl₃) δ=8.37 (dd, J=2.2, 7.7 Hz, 1H), 8.13 (d, J=8.3 Hz, 2H), 7.79 (s, 1H), 7.74 (d, J=8.3 Hz, 2H), 7.53 (ddd, J=1.2, 2.2, 7.7 Hz, 1H), 7.41 (m, 2H). MS calcd. for C₁₆H₁₀ClN₂S (M+H⁺) 297.0. found 296.8.

Step B: 4-(2-(2-Chlorophenyl)thiazol-4-yl)benzonitrile 2 (0.10 g, 0.3 mmol) is dissolved in DMF (1.50 mL). Sodium azide (0.09 g, 1.4 mmol) and ammonium chloride (0.09 g, 1.7 mmol) are added and the mixture is stirred at 110° C. for 18 h. Cooling, filtration, and reversed-phase HPLC purification (water/ACN gradient using TFA) yielded 5-(4-(2-(2-chlorophenyl)thiazol-4-yl)phenyl)-1H-tetrazole A1 as a white solid. MS calcd. for C₁₆H₁₁ClN₅S (M+H⁺) 340.0. found 340.0.

Table 1 shows illustrative compounds prepared following procedures described above, using appropriate starting materials that will be apparent to those skilled in the art.

TABLE 1

| | | Physical Data |
|---|---|---|
| | Compound | MS (m/z) and/or ¹H NMR |
| | Structure | (dmso-d₆, 400 MHz, δ) |
| A1 | 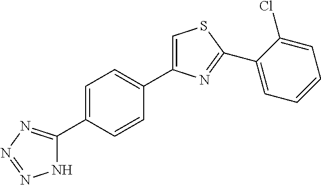 | MS calcd. for C₁₆H₁₁ClN₅S (M + H⁺) 340.0, found 340.0. |
| A2 | 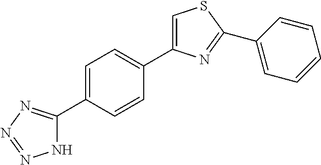 | MS calcd. for C₁₆H₁₂N₅S (M + H⁺) 306.1, found 305.8; ¹H-NMR (400 MHz, CDCl₃) δ = 8.39 (s, 1 H), 8.30 (d, J = 8.3 Hz, 2 H), 8.17 (d, J = 8.2 Hz, 2 H), 8.07 (m, 2 H), 7.57 (m, 3 H). |
| A3 | 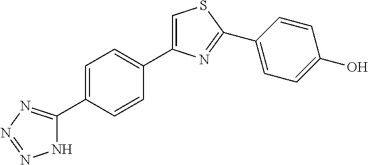 | MS calcd. for C₁₆H₁₂N₅OS (M + H⁺) 322.1, found 321.8; ¹H-NMR (400 MHz, CDCl₃) δ = 10.09 (s, 1 H), 8.27 (d, J = 8.5 Hz, 2 H), 8.25 (s, 1 H), 8.15 (d, J = 8.4 Hz, 2 H), 7.99 (d, J = 8.7 Hz, 2 H), 6.92 (d, J = 8.7 Hz, 2 H). |
| A4 | 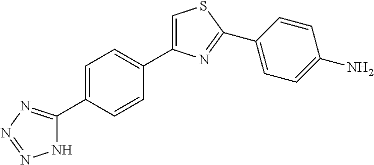 | MS calcd. for C₁₆H₁₃N₆S (M + H⁺) 321.1, found 320.8; ¹H-NMR (400 MHz, CDCl₃) δ = 8.26 (d, J = 8.5 Hz, 2 H), 8.15 (s, 1 H), 8.14 (d, J = 8.5 Hz, 2 H), 7.76 (d, J = 8.6 Hz, 2 H), 6.71 (d, J = 8.6 Hz, 2 H). |
| A5 | 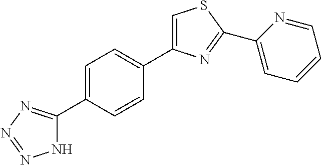 | MS calcd. for C₁₅H₁₁N₆S (M + H⁺) 307.1, found 306.8; ¹H-NMR (400 MHz, CDCl₃) δ = 8.68 (d, J = 4.6 Hz, 1 H), 8.48 (s, 1 H), 8.31 (m, 3 H), 8.17 (d, J = 8.4 Hz, 2 H), 8.04 (ddd, J = 1.6, 7.7, 7.7 Hz, 1 H), 7.56 (ddd, J = 1.0, 4.9, 7.5 Hz, 1 H). |
| A6 | 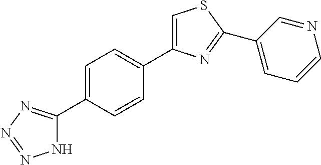 | MS calcd. for C₁₅H₁₁N₆S (M + H⁺) 307.1, found 306.8; ¹H-NMR (400 MHz, CDCl₃) δ = 9.31 (br. s, 1 H), 8.77 (br. s, 1 H), 8.49 (s, 1 H), 8.44 (d, J = 7.9 Hz, 1 H), 8.32 (d, J = 8.4 Hz, 2 H), 8.17 (d, J = 8.4 Hz, 1 H), 7.62 (br. s, 1 H). |
| A7 | 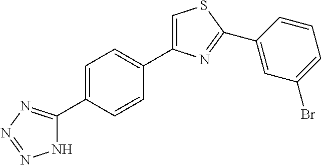 | MS calcd. for C₁₆H₁₁BrN₅S (M + H⁺) 384.0, found 383.9; ¹H-NMR (400 MHz, CDCl₃) δ = 8.44 (s, 1 H), 8.30 (d, J = 8.4 Hz, 2 H), 8.26 (dd, J = 1.8, 1.8 Hz, 1 H), 8.17 (d, J = 8.4 Hz, 2 H), 8.05 (ddd, J = 1.0, 1.6, 8.4 Hz, 1 H), 7.74 (ddd, J = 1.0, 1.6, 8.0 Hz, 1 H), 7.52 (dd, J = 7.9, 7.9 Hz, 1 H). |
| A8 | 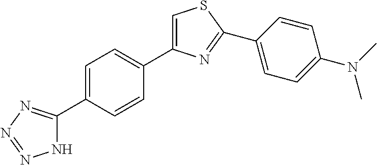 | MS calcd. for C₁₈H₁₇N₆S (M + H⁺) 349.1, found 349.0; ¹H-NMR (400 MHz, CDCl₃) δ = 8.26 (d, J = 7.8 Hz, 2 H), 8.17 (s, 1 H), 8.14 (d, J = 7.8 Hz, 2 H), 7.88 (d, J = 9.0 Hz, 2 H), 6.82 (d, J = 9.0 Hz, 2 H), 3.01 (s, 6 H). |

### Example 2

### 4-(4-(1H-Tetrazol-5-yl)phenyl)-N-(2-ethoxy-phenyl)thiazol-2-amine

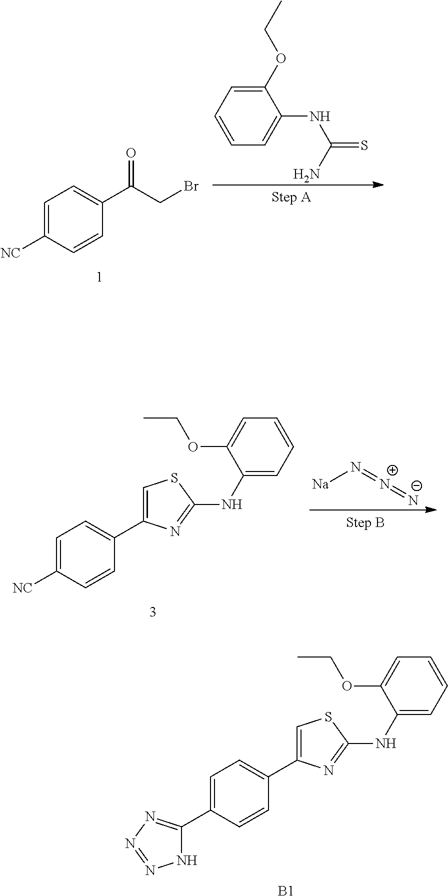

Step A: 4-(2-Bromoacetyl)benzonitrile 1 (0.022 g, 0.1 mmol) and 1-(2-ethoxyphenyl)thiourea (0.020 g, 0.1 mmol) are dissolved in EtOH (1 mL) and heated to 150° C. for 3 min using a microwave oven. Dilution with water, filtration, washing with water and drying afforded 4-(2-(2-ethoxyphenylamino)thiazol-4-yl)benzonitrile 3 as an off-white solid. MS calcd. for C₁₈H₁₆N₃OS (M+H⁺) 322.1. found 322.1.

Step B: 4-(2-(2-Ethoxyphenylamino)thiazol-4-yl)benzonitrile 3 (0.030 g, 0.1 mmol) is dissolved in DMF (1.50 mL). Sodium azide (0.02 g, 0.3 mmol) and ammonium chloride (0.02 g, 0.4 mmol) are added and the mixture was stirred at 110° C. for 18 h. Cooling, filtration, and reversed-phase HPLC purification (water/ACN gradient using TFA) yielded 4-(4-(1H-tetrazol-5-yl)phenyl)-N-(2-ethoxyphenyl)thiazol-2-amine B1 as a white solid. MS calcd. for C₁₆H₁₇N₆OS (M+H⁺) 365.1. found 365.1; ¹H NMR (dmso-d₆, 400 MHz): δ 9.51 (s, 1H), 8.50 (dd, J=2.3, 7.2 Hz, 1H), 8.13 (d, J=8.6 Hz, 2H), 8.09 (d, J=8.6 Hz, 2H), 7.53 (s, 1H), 7.01 (m, 3H), 4.14 (q, J=7.0, 2H), 1.41 (t, J=7.0 Hz, 3H).

Table 2 shows illustrative compounds prepared following procedures described above, using appropriate starting materials that will be apparent to those skilled in the art.

TABLE 2

| | | Physical Data |
|---|---|---|
| | Compound | MS (m/z) and/or ¹H NMR |
| | Structure | (dmso-d₆, 400 MHz, δ) |
| B1 | 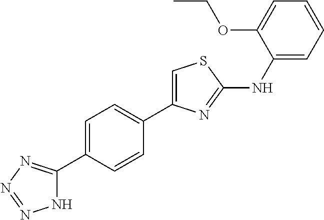 | MS calcd. for C₁₆H₁₇N₆OS (M + H⁺) 365.1, found 365.1; ¹H NMR (dmso-d₆, 400 MHz): δ 9.51 (s, 1 H), 8.50 (dd, J = 2.3, 7.2 Hz, 1 H), 8.13 (d, J = 8.6 Hz, 2 H), 8.09 (d, J = 8.6 Hz, 2 H), 7.53 (s, 1 H), 7.01 (m, 3 H), 4.14 (q, J = 7.0, 2 H), 1.41 (t, J = 7.0 Hz, 3 H). |
| B2 | 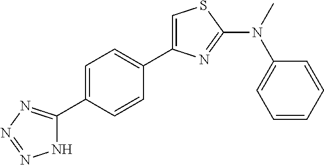 | MS calcd. for C₁₇H₁₅N₆S (M + H⁺) 335.1, found 335.2; 8.11 (d, J = 8.2 Hz, 1 H), ¹H- NMR (400 MHz, CDCl₃) δ = 8.08 (d, J = 8.2 Hz, 2 H), 7.55 (m, 2 H), 7.50 (m, 2 H), 7.32 (m, 1 H), 3.58 (s, 3 H). |
| B3 | 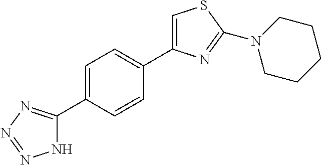 | MS calcd. for C₁₅H₁₇N₆S (M + H⁺) 313.1, found 313.2; 8.08 (d, J = 8.2 Hz, 1 H), ¹H- NMR (400 MHz, CDCl₃) δ = 8.06 (d, J = 8.2 Hz, 2 H), 7.44 (s, 1 H), 3.50 (br, 4 H), 1.63 (br, 6 H). |
| B4 | 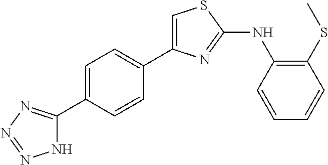 | MS calcd. for C₁₇H₁₅N₆S₂ (M + H⁺) 367.1, found 367.1; ¹H-NMR (400 MHz, CDCl₃) δ = 9.51 (s, 1 H), 8.07 (s, 4 H), 7.88 (d, J = 7.9 Hz, 1 H), 7.48 (s, 1 H), 7.40 (d, J = 7.7 Hz, 1 H), 7.28 (ddd, J = 1.4, 7.4, 7.4 Hz, 1 H), 7.20 (ddd, J = 1.0, 7.4, 7.6 Hz, 1 H), 2.44 (s, 3 H). |
| B5 | 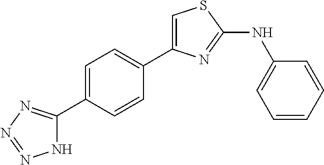 | MS calcd. for C₁₆H₁₃N₆S (M + H⁺) 321.1, found 321.1; ¹H-NMR (400 MHz, CDCl₃) δ = 10.37 (s, 1 H), 8.16 (d, J = 8.2 Hz, 2 H), 8.12 (d, J = 8.2 Hz, 1 H), 7.75 (d, J = 8.1 Hz, 2 H), 7.57 (s, 1 H), 7.38 (t, J = 7.4 Hz, 2 H), 7.00 (t, J = 7.3 Hz, 1 H). |
| B6 | 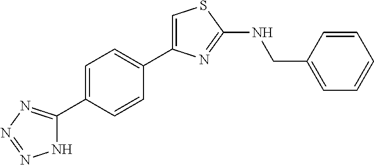 | MS calcd. for C₁₇H₁₅N₆S (M + H⁺) 335.1, found 335.0; ¹H-NMR (400 MHz, CDCl₃) δ = 8.32 (br, 1 H), 8.05 (s, 4 H), 7.41 (m, 2 H), 7.36 (m, 2 H), 7.30 (s, 1 H), 7.27 (m, 1 H), 4.55 (s, 2 H). |

### Example 3

### 5-(4-(4-phenylthiazol-2-yl)phenyl)-1H-tetrazole

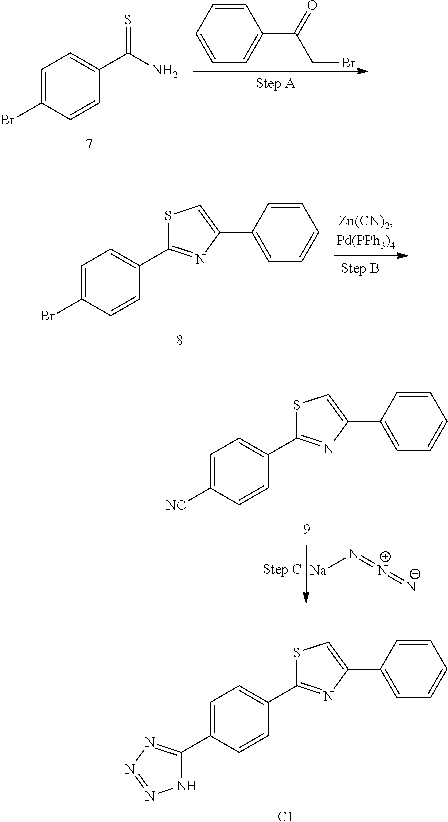

Step A: 4-Bromobenzothioamide 7 (0.02 g, 0.1 mmol) and 2-bromo-1-phenylethanone (0.02 g, 0.1 mmol) are dissolved in EtOH (1 mL) and heated to 150° C. for 3 min using a microwave oven. Dilution with water, filtration, washing with water and drying afforded 2-(4-bromophenyl)-4-phenylthiazole 8 as an off-white solid. MS calcd. for C₁₅H₁₁BrNS (M+H⁺) 316.0. found 315.8.

Step B: 2-(4-Bromophenyl)-4-phenylthiazole 8 (0.03 g, 0.1 mmol) zinc cyanide (0.05 g, 0.4 mmol) and tetrakis-triphenylphosphinepalladium(0) (0.01 g, 0.09 mmol) are dissolved in DMA (1 mL) and heated to 110° C. for 18 h. Dilution with water, extraction with ethyl acetate, washing with water and brine, drying over MgSO₄ and concentration afforded 4-(4-phenylthiazol-2-yl)benzonitrile 9, which was used in the next step without further purification. MS calcd. for C₁₆H₁₁N₂S (M+H⁺) 263.1. found 263.0.

Step C: Crude 4-(4-phenylthiazol-2-yl)benzonitrile 9 (from Step B above) is dissolved in DMF (1 mL). Sodium azide (0.02 g, 0.3 mmol) and ammonium chloride (0.02 g, 0.4 mmol) are added and the mixture was stirred at 110° C. for 18 h. Cooling, filtration, and reversed-phase HPLC purification (water/ACN gradient using TFA) yielded 5-(4-(4-phenylthiazol-2-yl)phenyl)-1H-tetrazole C1 as an oil. MS calcd. for C₁₆H₁₂N₅S (M+H⁺) 306.1. found 306.1.

Table 3 shows illustrative compounds prepared following procedures described above, using appropriate starting materials that will be apparent to those skilled in the art.

TABLE 3

| | | Physical Data |
|---|---|---|
| | Compound | MS (m/z) and/or ¹H NMR |
| | Structure | (dmso-d₆, 400 MHz, δ) |
| C1 | 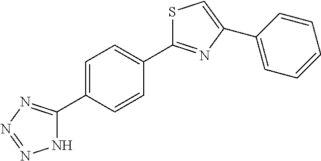 | MS calcd. for C₁₆H₁₂N₅S (M + H⁺) 306.1, found 306.1. |
| C2 | 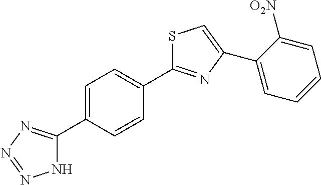 | MS calcd. for C₁₆H₁₁N₆O₂S (M + H⁺) 351.1, found 351.0. |
| C3 | 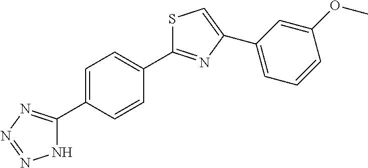 | MS calcd. for C₁₇H₁₄N₅OS (M + H⁺) 336.1, found 336.1; ¹H-NMR (400 MHz, CDCl₃) δ = 8.33 (s, 1 H), 8.28 (d, J = 8.4 Hz, 2 H), 8.22 (d, J = 8.4 Hz, 2 H), 7.67 (ddd, J = 1.0, 1.0, 8.4 Hz, 1 H), 7.20 (dd, J = 1.0, 7.8 Hz, 1 H), 7.64 (dd, J = 1.0, 1.5 Hz, 1 H), 7.42 (dd, J = 7.9, 8.0 Hz, 1 H), 6.98 (ddd, J = 1.0, 1.5, 8.2 Hz, 1 H), 3.86 (s, 3 H). |
| C4 | 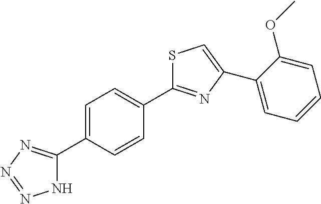 | MS calcd. for C₁₇H₁₄N₅OS (M + H⁺) 336.1, found 336.1; ¹H-NMR (400 MHz, CDCl₃) δ = 8.34 (dd, J = 1.8, 7.8 Hz, 1 H), 8.28 (d, J = 8.5 Hz, 2 H), 8.25 (s, 1 H), 8.21 (d, J = 8.5 Hz, 2 H), 7.40 (ddd, J = 1.8, 7.3, 8.4 Hz, 1 H), 7.20 (dd, J = 1.0, 8.4 Hz, 1 H) 7.11 (ddd, J = 1.0, 7.6, 7.6 Hz, 1 H), 3.97 (s, 3 H). |
| C5 | 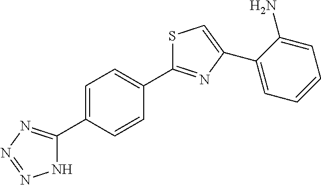 | MS calcd. for C₁₆H₁₃N₆S (M + H⁺) 321.1, found 320.9. |
| C6 | 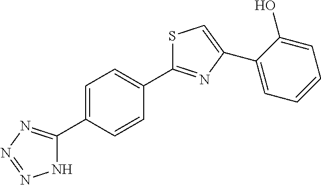 | MS calcd. for C₁₆H₁₂N₆OS (M + H⁺) 322.1, found 322.0. |
| C7 | 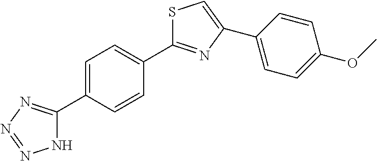 | MS calcd. for C₁₇H₁₄N₅OS (M + H⁺) 336.1, found 336.1; ¹H-NMR (400 MHz, CDCl₃) δ = 8.27 (d, J = 8.6 Hz, 2 H), 8.21 (d, J = 8.6 Hz, 2 H), 8.13 (s, 1 H), 8.03 (d, J = 8.8 Hz, 2 H), 7.06 (d, J = 8.8 Hz, 1 H), 3.83 (s, 3 H). |

### Example 4

### N-(4-((1H-Tetrazol-5-yl)methoxy)phenyl)-4-(2-chlorophenyl)thiazol-2-amine

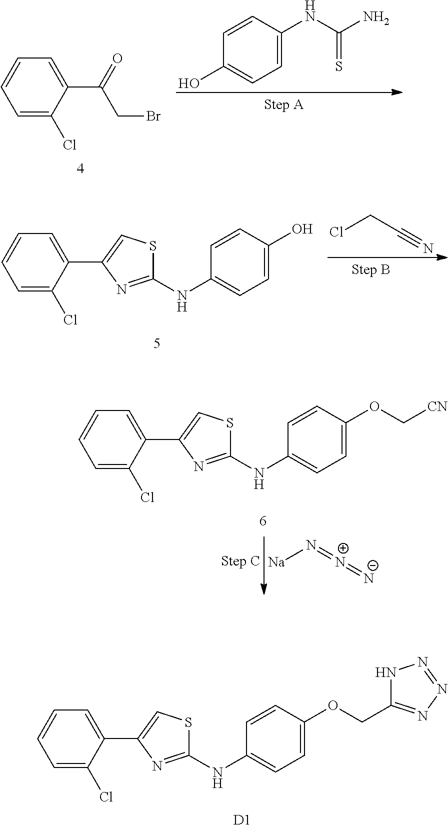

Step A: 2-Bromo-1-(2-chlorophenyl)ethanone 4 (0.23 g, 1.0 mmol) and 1-(4-hydroxyphenyl)thiourea (0.15 g, 1.0 mmol) are dissolved in EtOH (3 mL) and heated to 150° C. for 3 min using a microwave oven. Dilution with water, filtration, washing with water and drying afforded 4-(4-(2-chlorophenyl)thiazol-2-ylamino)phenol 5 as a yellow solid. ¹H-NMR (400 MHz, dmso-d₆) δ=10.04 (s, 1H), 8.01 (s, 1H), 7.97 (dd, J=2.0, 7.6 Hz, 1H), 7.84 (d, J=8.7 Hz, 2H), 7.59 (dd, J=1.5, 7.7 Hz, 1H), 7.47 (ddd, J=1.5, 7.7, 7.7 Hz, 1H), 7.42 (ddd, J=2.0, 7.7, 7.7 Hz, 1H), 6.90 (d, J=8.7 Hz, 2H). MS calcd. for C₁₅H₁₁ClNOS (M+H⁺) 288.1. found 288.0.

Step B: 4-(4-(2-Chlorophenyl)thiazol-2-ylamino)phenol 5 (0.032 g, 0.11 mmol) and chloroacetonitrile (0.015 mL, 0.24 mmol) are dissolved in ACN (1.5 mL). Cesium carbonate (0.05 g, 0.15 mmol) is added and the mixture is stirred at 60° C. for 3 h. Filtration, washing the solids with more ACN, drying the combined organic extracts over Na₂SO₄ and concentration afforded 2-(4-(4-(2-chlorophenyl)thiazol-2-ylamino)phenoxy)acetonitrile 6 as an oil. MS calcd. for C₁₇H₁₂ClN₂OS (M+H⁺) 327.0. found 326.7.

Step C: 2-(4-(4-(2-chlorophenyl)thiazol-2-ylamino)phenoxy)acetonitrile 6 (0.027 g, 0.08 mmol) is dissolved in DMF (1.0 mL). Sodium azide (0.02 g, 0.3 mmol) and ammonium chloride (0.02 g, 0.4 mmol) are added and the mixture is stirred at 110° C. for 18 h. Cooling, filtration, and reversed-phase HPLC purification (water/ACN gradient using TFA) yielded N-(4-((1H-tetrazol-5-yl)methoxy)phenyl)-4-(2-chlorophenyl)thiazol-2-amine D1 as a white solid. MS calcd. for C₁₇H₁₃ClN₅OS (M+H⁺) 370.1. found 369.7.

Assays

The suitability of a compound to modulate G protein-coupled receptor 120 (GPR120) may be tested following the assays described below, or using methods known in the art, such as those described in EP 1688138, incorporated herein by reference in its entirety.

Generation of GPR120-Expressing Cells

Human GPR120 stable cell-line was generated in HE 293 cells. GPR120 (Accession number BC101175) is fused to a promiscuous G protein, Gα16. The expression plasmid is transfected into HEK293 cells using Fugene6 following manufacturer's instruction. Stable cell-lines are generated following drug selection.

FLIPR Assay

FLIPR (Fluorimetric Imaging Plate Reader, Molecular Devices) assays are performed to measure agonist-induced calcium mobilization in the GPR120-expressing cells. One day before the FLIPR assay, HEK293-GPR120-Ga16 cells are seeded into poly-D-lysine coated black-wall-clear bottom 384 well plates (Falcon) at 25,000 cells per well in 40 μl DMEM supplemented with 1% of FBS. The cells are incubated overnight at 37° C. in a humidified incubator. The medium is aspirated on the day of the FLIPR assay. The cells are incubated with 50 μl/well of the assay buffer (HBSS, 20 mM HEPES, 2.5 mM probenecid, pH7.4) containing Fluo-4 NM dye (Invitrogen cat#F36205) at 37° C. for 45 minutes, and then equilibrated at room temperature for 30 minutes. Compounds are dissolved in DMSO and diluted to desired concentrations with assay buffer. Fluorescent output is measured immediately following compound addition (12.5 μl/well) on the FLIPR machine.

An EC₅₀ value was calculated using the change of fluorescent intensity from the reaction initiation. In general, compounds of the invention may have EC₅₀ values of 10 μM or less, for example from 0.1 μM to 10 μM. In some examples, compounds of the invention may have EC₅₀ values from 0.1 μM to 5 μM; or more particularly from 0.5 μM to 5 μM. In other examples, compounds of the invention may have EC₅₀ values of 3 μM or less; for example from 0.1 μM to 3 μM. In yet other examples, compounds may have EC₅₀ values less than 0.1 μM or more than 10 μM.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1. A compound of Formula (1A) or (1B): 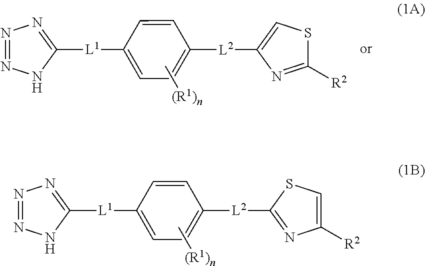
or pharmaceutically acceptable salts thereof, wherein:
L¹ is a bond or —CR³₂—O—;
L² is a bond or NR³;
R¹ is OR, or an optionally substituted C₁₋₆ alkyl, C₂₋₆ alkenyl or C₃₋₆ alkynyl;
R² is NR³—(CR³₂)ₘR⁴ or R⁴;
R³ is H or C₁₋₆ alkyl;
R⁴ is C₃₋₇ cycloalkyl, aryl, heteroaryl or heterocyclic ring containing N, O or S, each of which is optionally substituted with halo, hydroxyl, nitro, OR, SR, NR₂, NR³—(CR³₂)ₘ—R⁴, or an optionally substituted C₁₋₆ alkyl, C₂₋₆ alkenyl or C₃₋₆ alkynyl; or
R³ and —(CR³₂)ₘ—R⁴ together with N in NR³—(CR³₂)ₘ—R⁴ form an optionally substituted heterocyclic ring;
each R is H, an optionally substituted C₁₋₆ alkyl or —(CR³₂)ₘ—R⁴; and
m and n are independently 0-4.

2. The compound of claim 1, wherein said compound is of Formula (2A) or (2B): 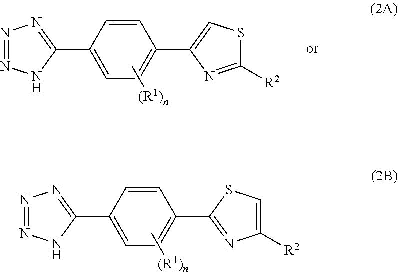
wherein each n is 0.

3. The compound of claim 1, wherein R² is an optionally substituted phenyl, piperidinyl or pyridyl.

4. The compound of claim 1, wherein said compound is of Formula (3A) or (3B): 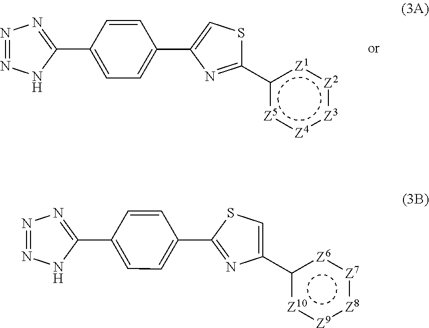
wherein Z¹, Z², Z³, Z⁴, Z⁵, Z⁶, Z⁷, Z⁸, Z⁹ and Z¹⁰ are independently CR⁵ or N; and
R⁵ is H halo, hydroxyl, nitro, OR, SR, NR₂, NR³—(CR³₂)ₘ—R⁴, or an optionally substituted C₁₋₆ alkyl, C₂₋₆ alkenyl or C₃₋₆ alkynyl.

5. The compound of claim 4, wherein each Z¹, Z², Z³, Z⁴, Z⁵, Z⁶, Z⁷, Z⁸, Z⁹ and Z¹⁰ is CR⁵.

6. The compound of claim 4, wherein one of Z¹, Z², Z³, Z⁴, Z⁵, Z⁶, Z⁷, Z⁸, Z⁹ and Z¹⁰ is N.

7. The compound of claim 1, wherein said compound is of Formula (4A) or (4B): 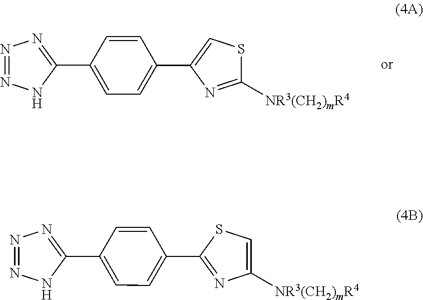
wherein R³ is H or C₁₋₆ alkyl;
R⁴ is a 5-6 membered aryl or heteroaryl; or
R³ and —(CR³₂)ₘ—R⁴ together with N in NR³—(CR³₂)ₘ—R⁴ form an optionally substituted 4-7 membered heterocyclic ring; and
m is 0 or 1.

8. The compound of claim 7, wherein R⁴ is phenyl or R³ and —(CR³₂)ₘ—R⁴ together with N in NR³—(CR³₂)ₘ—R⁴ form an optionally substituted piperidinyl.

9. A pharmaceutical composition comprising a therapeutically effective amount of a compound according to claim 1.

10-15. (canceled)

16. A method for modulating G protein-coupled receptor 120 (GPR120) comprising administering to a cell or tissue system, a therapeutically effective amount of a compound of claim 1, or a pharmaceutically acceptable salt or pharmaceutical composition thereof, and optionally in combination with a second therapeutic agent, thereby modulating said GPR120.

17. The method of claim 16, wherein said compound is a GPR120 agonist.

18. A method for treating a condition mediated by G protein-coupled receptor 120 (GPR120), comprising administering to a mammalian subject, an effective amount of a compound of claim 1, or a pharmaceutically acceptable salt or pharmaceutical composition thereof, and optionally with a second therapeutic agent, thereby treating said condition.

19. The method of claim 18, wherein said condition is diabetes, dyslipidemia, obesity or anorexia.

20. The method of claim 19, wherein said diabetes is diabetes mellitus.

21. The method of claim 19, wherein said dyslipidemia is hyperlipidemia.
